(19) United States
(12) Patent Application Publication
(10) Pub. No.: US 2007/0010477 A1
(43) Pub. Date: Jan. 11, 2007

(54) Acyl homoserine lactones for inhibition of cell growth

(76) Inventors: Bruce J. Dolnick, Orchard Park, NY (US); Janice R. Sufrin, Snyder, NY (US); Norman J. Angelino, Amherst, NY (US); Ree Y. Dolnick, Orchard Park, NY (US); Lawrence V. Stephanie, Rochester, MN (US); Colin M. Oliver, Buffalo, NY (US)

Correspondence Address: HODGSON RUSS LLP, ONE M & T PLAZA, SUITE 2000, BUFFALO, NY, 14203-2391, US

(21) Appl. No.: 11/399,933

(22) Filed: Apr. 7, 2006

Related U.S. Application Data
(60) Provisional application No. 60/669,279, filed on Apr. 7, 2005.

(51) Int. Cl. A61K 31/7072; A61K 31/513; A61K 31/365; A61K 31/337

(52) U.S. Cl. 514/49; 514/471; 514/473; 514/449; 514/269

(57) ABSTRACT

The present invention provides a method for inhibiting the growth of cancer cells using AHLs of the general formula CX-homoserine lactone where “X” represents a number of between 5 and 14 carbon atoms in the acyl chain of the AHL. The method comprises the step of administering to an individual an amount of an AHL effective to inhibit the growth of cancer cells. Also provided is a method for enhancing the effect of a chemotherapeutic agent comprising the step of administering to an individual the chemotherapeutic agent and an amount of an AHL effective to enhance the cancer cell growth inhibitory effect of the chemotherapeutic agent.

This application claims priority to U.S. provisional application Ser. No. 60/669,279, filed Apr. 7, 2005, the disclosure of which is incorporated herein by reference.

This work was supported by NIH grants CA86876, CA57634, CA80684 and CA16056. The government has certain rights in the invention.

### FIELD OF THE INVENTION

This invention relates generally to the area of cancer and more particularly to the use of acyl homoserine lactones (AHLs) to inhibit cell growth and to enhance the effect of chemotherapeutic agents.

### BACKGROUND OF THE INVENTION

Thymidylate synthase (TS) is an essential enzyme involved in the synthesis of the DNA base thymidine 5′monophosphate (dTMP) from the RNA base deoxyuridine 5′-monophosphate (dUMP). TS is required for DNA biosynthesis and repair, has been shown to function as an oncogene (Rahman, et al. Cancer Cell. 2004, 4: 341-351) and continues to be recognized as an important target for anticancer agents. This is attested to by the large number of TS inhibitors introduced over the last few decades, particularly fluorinated pyrimidines, metabolites of which (including metabolites of 5-fluorouracil and 5-fluorodeoxyuridine) compete with deoxyuridylate for binding to TS. However, a major problem with these types of compounds is either non-responsiveness of the tumor cell or the development of resistance. A common mechanism cancer cells use to develop resistance is increased expression of TS.

The TS mRNA has a region complementary to the RNA from a gene termed rTS (also known as ENOSF1). The rTS gene overlaps the 3′-end of the TS gene on chromosome 18 such that the two genes produce partially complementary RNA transcripts (Dolnick, 1993, Nuc. Acids Res. 21:1747-52). The rTS gene encodes for at least two products produced by alternative splicing. rTSβ is the major protein product, while rTSα is a minor protein product produced in small amounts as a protein but in large amounts as heterogeneous nuclear RNA (Dolnick, 1993, Nuc. Acids Res. 21, 1747-52; Dolnick et al., 1996, Cancer Res. 56:3207-3210). While all the functions of these rTS products remains unclear, research indicates that rTSβ is likely involved in the production of secreted lipophilic compounds derived from methionine that can cause the downregulation of TS in cell cultures (Dolnick et al. (2003) Cancer Biol. Ther. 2:364-9). Further, since rTS is widely expressed in human tissue, and its expression is altered in some cell lines resistant to anticancer drugs, it is possible that rTS plays a role in cell growth in vivo and that the expression of the rTS-associated secreted lipophilic compounds can affect response to cancer drug treatment. Therefore, there is a need to characterize the secreted lipophilic compounds and to develop methods for using such compounds for inhibiting cancer cell growth.

### SUMMARY OF THE INVENTION

The present invention is based on the discovery that human cancer cells which over-express rTSβ secrete one or more compounds capable of causing down regulation of TS expression, that the conditioned medium of these cells contains AHLs, that rTSβ is involved in the synthesis of AHLs, and that AHLs can downregulate TS. Based on these unexpected findings, the present invention provides a method for inhibiting the growth of cancer cells in an individual. The method comprises the step of administering to the individual a composition comprising an amount of an AHL effective to inhibit the growth of the cancer cells. It is demonstrated that the administration of AHLs can inhibit the growth of a variety of cancer cells, including human colorectal, lung, breast and prostate cancer cells.

AHLs useful in the method of the invention are those of the L form and which have the general formula of CX-homoserine lactone, where “X” represents a number of between 5 and 14 carbon atoms in the acyl chain of the AHL. Certain positions of the AHLs can also be modified, such as by addition of an aromatic group to the acyl chain, without adversely affecting their capacity to inhibit cancer cell growth. It is preferred that the AHLs are 3-oxo-CX-homoserine lactones.

Also provided is a method for enhancing the effect of a chemotherapeutic agent. The method comprises the step of administering to an individual a chemotherapeutic agent and an amount of an AHL effective to enhance the cancer cell growth inhibitory effect of the chemotherapeutic agent relative to administration of the chemotherapeutic agent alone. In particular, it is shown that AHLs can enhance the effect of chemotherapeutic agents known to predominantly target TS, as well as the effects of chemotherapeutic agents thought to act in a manner unrelated to TS expression, such as microtubule inhibitors. Moreover, AHLs can enhance the activity of microtubule inhibitors against cells which are known to be resistant to chemotherapeutic agents that affect TS.

### BRIEF DESCRIPTION OF THE DRAWINGS

FIG. 1 is a graphical representation of down-regulation of TS by diffusible products produced by H630-1 human colon cancer cells (over-expressers of rTSβ) or H630 human colon cancer cells as detected by immunoblotting with antibodies for rTSβ, TS and tubulin. The triangle at the top of the figure shows the increasing density of the cells from which the extracts were prepared for analysis.

FIG. 2A shows a graphical representation of relative light units (RLUs) measured in a luminescence bioassay using 3-(oxo)-C6 homoserine lactone, the natural signaling molecule for the bacterial receptor used in this assay (FIG. 1A). FIG. 2B demonstrates the presence of molecules extracted from conditioned (i.e., spent) medium from H630-1 cells (•), H630 cells (∘) or control (Δ) expressed as relative light units, and the relative high abundance of these molecules in medium from H630-1 cells compared to H630 cells or control medium. FIG. 2C is a graphical representation of an ¹H-NMR analysis of extract from spent media of H630-1 cells results confirming the presence of an AHL. The top spectrum is an analysis of the extract. The bottom spectrum is an analysis of a homoserine lactone with the ring protons labeled as shown (H).

FIGS. 3A through FIG. 3D are graphical representations of plots of radioactivity in HPLC fractions of extracted spent medium from H630 cells (FIG. 3A) and H630-1 cells (FIGS. 3B, 3C, 3D) showing methionine metabolites containing [¹⁴COOH]-methionine (FIG. 3A and FIG. 3B), [¹⁴CH₃]-methionine (FIG. 3C) and [³⁵S]-methionine (FIG. 3D).

FIG. 4 is a graphical representation of the enzyme activity by refolded rTSβ. The inset depicts a photographic representation of a Coomassie stained denaturing polyacrylamide gel with the preparations of rTSβH6 (left) and rTSαH6 (right) used for the assay.

FIG. 5 is a representation of the effect of various AHLs on TS, rTSβ and tubulin. The indicated AHLs are (top to bottom: 3-hydroxy-butanoyl homoserine lactone, butanoyl homoserine lactone, 3-(oxo)-hexanoyl homoserine lactone, hexanoyl-homoserine lactone, hexanoyl-(L)-homoserine lactone) and 3-(oxo)-ethyl hexanoic acid.

FIG. 6 is a photographical representation of Western blot analysis of a time course for downregulation of TS protein treated with 3-oxo-hexanoyl(C6)-L-homoserine lactone (3 mM).

FIGS. 7A and 7B are photographic representations of Western blot analysis showing the distribution of TS and rTS gene products in the indicated tissues.

FIGS. 8A and FIG. 8B depict Western blots and corresponding graphical analysis of the effects of AHLs on TS and tubulin in H630 cells. FIG. 8A depicts the effects of short-chain AHLs. The AHLs (top to bottom) are: 3-OH—C4-HSL, C4-HSL, 3-OH—C5-HSL, C5-HSL, 3-oxo-C6-HSL and the last two compounds are C6-HSL as the racemic (D, L) or (L) isomer. The compound without a lactone ring (x), shown on the bottom serves as a control for the 3-oxo-C6-HSL. Each lane was loaded with 20 μg of protein. TS was detected with polyclonal antibody. Tubulin (Tb) was used as a marker for protein loading. Solid bars indicate TS amounts. FIG. 8B depicts the effects of C12 HSLs on TS in H630 cells. Results for tubulin are included to illustrate the changes observed with 3-oxo-C12-L-HSL. Expression of TS and/or tubulin is displayed in amounts relative to expression in untreated cells. Solid bars indicate TS; shaded columns indicate tubulin. FIG. 8C is a photographic representation of an eletrophoretic analysis of the effect of 23 μM 3-oxo-C12-HSL on TS mRNA levels.

FIGS. 9A and 9B are graphical representations of the effect of two AHLs on cell growth. FIG. 9A depicts the activity of 3-oxo-C6-HSL and 3-oxo-C12-HSL in the TS modulation assay. RLU, relative light units. FIG. 9B depicts a comparison of various AHLs on the growth of H630 cells. Solid circles (•), 3-oxo-C12-HSL; Open circles (∘), 3-oxo-C12-(D,L)—HSL; Closed triangles (▴), C12-HSL; Open triangles (∇), 3-oxo-C6-HSL. FIG. 9C is a graphical representation of cell growth inhibition for 3-oxo-C12-HSL in MCF-7 cells, a breast cancer cell line, and NCI—H460, a lung cancer cell line. The IC50 values are 16 μM for MCF-7 and 35 μM r for NCI—H460.

FIG. 10 is a representation of the ehancement of 5-fluorouracil (FU) inhibition of colony formation by 3-oxo-C12-HSL. Either H630 or H630-1 cells were allowed to form colonies in the presence of FU, either by itself (•) or in combination (∘) with an IC50 amount of 3-oxo-C12-HSL (23 μM for H630 and 8 μM for H630-1) and counted after 2 weeks. A sample photograph of results for a subset of drug concentrations in H630 cells is shown on the right. The * next to the top plate signifies that the two samples in the left column were accidentally reversed at the time of drug addition.

FIGS. 11A through 11F are graphical representations of the interactions of 3-oxo-C12-HSL with chemotherapeutic agents. Cells were plated in the presence of the indicated compounds, with or without 3-oxo-C12-HSL (23 or 8 μM). Colonies were counted after approximately 2 weeks. Each treatment was performed with H630 cells (top panels) and H630-1 cells (bottom panels).

FIGS. 12A and 12B are photographic representations of the morphology of H630 and H630-1 cells. Colonies were allowed to form (FIG. 12A, H630; FIG. 12B, H630-1) and were stained with methylene blue and photographed using phase contrast microscopy. The bar in panel A represents 10 μ in length.

FIGS. 13A through 13C are graphical representations of cell growth inhibition data of various length AHLs modified with phenyl groups. FIG. 13A presents data for H630 cells. FIG. 13B present data for H630-1 cells. FIG. 13C present data for PC-3 (prostate cancer) cells. FIG. 13D depicts the phenyl modified AHLs used to obtain the data presented in FIGS. 13A-13C.

### DETAILED DESCRIPTION OF THE INVENTION

This invention is based on several observations relating to TS. It was observed that cells which over-express rTSβ, secreted one or more products capable of causing down regulation of TS expression (Example 2). The conditioned medium of these cells was found to contain AHLs (Examples 3 and 4). AHLs were also found to downregulate TS (Example 7). Further, rTSβ was implicated in the synthesis of AHLs (Example 5). Based on these surprising observations, the present invention provides a method for inhibiting the growth of cancer cells comprising the step of administrating to an individual an amount of an AHL effective to inhibit the growth of the cancer cells. The AHLs are shown to be effective at inhibiting the growth of a variety of cancer cell types and are effective for inhibiting the growth of all cancers, including solid tumors.

Also provided is a method for enhancing the effect of chemotherapeutic agents. The method comprises the step of administering, in combination with a chemotherapeutic agent, an amount of an AHL effective to enhance the growth inhibitory effect of the chemotherapeutic agent relative to administration of the chemotherapeutic agent alone. In particular, AHLs are shown to enhance the growth inhibitory effect of conventional chemotherapeutic agents known to target TS, such as 5-fluorouracil (FU), fluorodeoxyuridine (FUdR) and tomudex. This effect is demonstrated in a human cancer cell line. The use of cancer cell line models to predict the in vivo enhancement of chemotherapeutic agents has been established and is well recognized by those skilled in the art. For example, calcium leucovorin has previously been shown to similarly enhance FU activity against cancer cells in vitro, and subsequently in vivo (see, Martindale: The Complete Drug Reference (33rd edition); Sweetman et al. Pharmaceutical Press, 2002). Therefore, the use of cancer cell line models to predict the in vivo enhancement of chemotherapeutic agents has been established. AHLs also enhance the activity of chemotherapeutic agents that act on targets thought to be unrelated to TS, such as the microtubule inhibitor taxol. The enhancement of the activity of taxol is further demonstrated to be effective in human cancer cells that are resistant to FU. Thus, the method of the invention is useful for treating a variety of cancers using various chemotherapeutic regimens.

AHLs for use in the method of the invention are of the L form and have the general formula of CX-homoserine lactone, where “X” represents a number of between 5 and 14 carbon atoms in the acyl chain of the homoserine lactone. In preferred embodiments, the AHLs are 3-oxo-CX-homoserine lactones. A preferred 3-oxo-CX-homoserine lactone is 3-oxo-C12 homoserine lactone. The AHLs can also be modified, such as by addition of an aromatic group, without adversely affecting their capacity to inhibit cancer cell growth.

In more detail, the AHLs useful for the present invention have the following general formula:

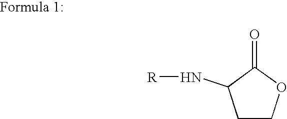

Wherein R is an acyl group represented by: 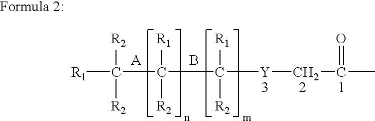
wherein Y may be C═O, C═S, C═NH, CHOH, CHSH, CH₂, or R₁—C—R₂;

R₁ may be independently at each occurrence H, alkyl, Ar or CH₂, wherein Ar is an aromatic substitution;

R₂ may be independently at each occurrence H, F, Cl, Br, or I;

n and m can each independently be between and include 0-10, and the sum of n and m is between and includes 1 and 10; and

A and B indicate positions where a group R₃ may be introduced, wherein R₃ is CH═CH. As used herein, “acyl chain” means the formula depicted in Formula 2. The numbering of carbon atoms shown in Formula 2 (numbers 1, 2 and 3) is provided for convenience in referring to the various AHLs used in the method of the invention. The carbonyl carbon labeled “1” in Formula 2 is the Cl of the acyl chain.

Other homoserine lactones useful for use in the present invention can be identified using the methods described herein. For example, AHLs or AHL analogs can be tested in cancer cell models, such as the human colorectal cancer cell line H630, and the TS and rTSβ-overproducing, 5-FU resistant human colorectal cancer cell line H630-1.

AHLs useful for the present invention can be synthesized by coupling L-homoserine lactone with a carboxylic acid of general structure: 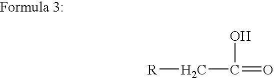
in the presence of a coupling reagent from the structural class of carbodiimides. The general preparation of acyl homoserine lactones using this synthetic method is outlined in detail by Camara et al. (1998, Methods in Microbiology 27, 319-330). The general procedure for synthesizing N-acyl homoserine lactones outlined by Camara et al. (1998) is patterned after methodology for synthesis of amide structures. This same methodology has been widely used for the stepwise synthesis of peptides.

Whereas Camara et al. (1998) have outlined a general synthesis of N-acyl homoserine lactones which entails coupling of a carboxylic acid with L-homoserine lactone, for the present invention, a modification of this method has been used whereby the sodium salt of a carboxylic acid in place of a carboxylic acid is used. Specific procedures used for the synthesis of N— AHLs and 3-oxo-N AHLs are provided in the Examples.

The AHLs may be combined with pharmaceutically acceptable carriers to form compositions for use in inhibiting cell growth and/or for enhancing the activity of a chemotherapeutic agent. Acceptable pharmaceutical carriers are described in Remington's Pharmaceutical Sciences (18th Edition, A. R. Gennaro et al. Eds., Mack Publishing Co., Easton, Pa., 1990). Compositions comprising AHLs are preferably homogeneous as to the L form of the AHLs, as data demonstrate racemic mixtures are less effective at inhibiting cancer cell growth, while the D form is largely ineffective.

In one embodiment, the AHL can be provided in combination with a pharmaceutically appropriate delivery composition. One example of a suitable vehicle composition is nanoparticles. A variety of suitable nanoparticles are known in the art, such as PLA (poly-lactic acid) and PGA (polyglycolic acid). A preferred composition for nanoparticles is the clinically approved compound lactic-co-glycolic acid (PLGA), preferably D, L-lactic-co-glycolic acid. The nanoparticles can be formed by polymerization using conventional techniques. AHLs can be incorporated into the nanoparticles by polymerizing the nanoparticles in the presence of the AHL. Preferably, the nanoparticles are of a diameter of between 70-100 nm. It will be recognized by one of skill in the art that the form and character of the pharmaceutically acceptable carrier will be dictated by the amount of AHL with which it is to be combined, the route of administration, the nature of the tumor and other well-known variables.

When the compositions comprising AHLs further comprise chemotherapeutic agents for administration to an individual such that the cancer cell growth inhibitory effect of the chemotherapeutic agent is enhanced. Suitable chemotherapeutic agents for administration with the AHLs include those that affect TS, such as 5-fluorouracil (FU), fluorodeoxyuridine (FUdR) and tomudex, and those that affect microtubules, such as taxol and paclitaxel.

Compositions comprising AHLs in an amount effective to inhibit the growth of a cancer cell and/or to enhance the cancer cell growth inhibition activity of a co-administered chemotherapeutic agent can be administered to an individual using various methods known to those skilled in the art. These methods include intradermal, intramuscular, intraarterial, intrathecal, oral, intraperitoneal, intravenous, subcutaneous, and intranasal routes. Further, those skilled in the art will recognize that the dosage of the AHLs will depend on well known variables, such as the size of the individual and the stage of the cancer.

The following examples are provided to better understand the invention and are intended to be illustrative and are not to be construed as restrictive.

### EXAMPLE 1

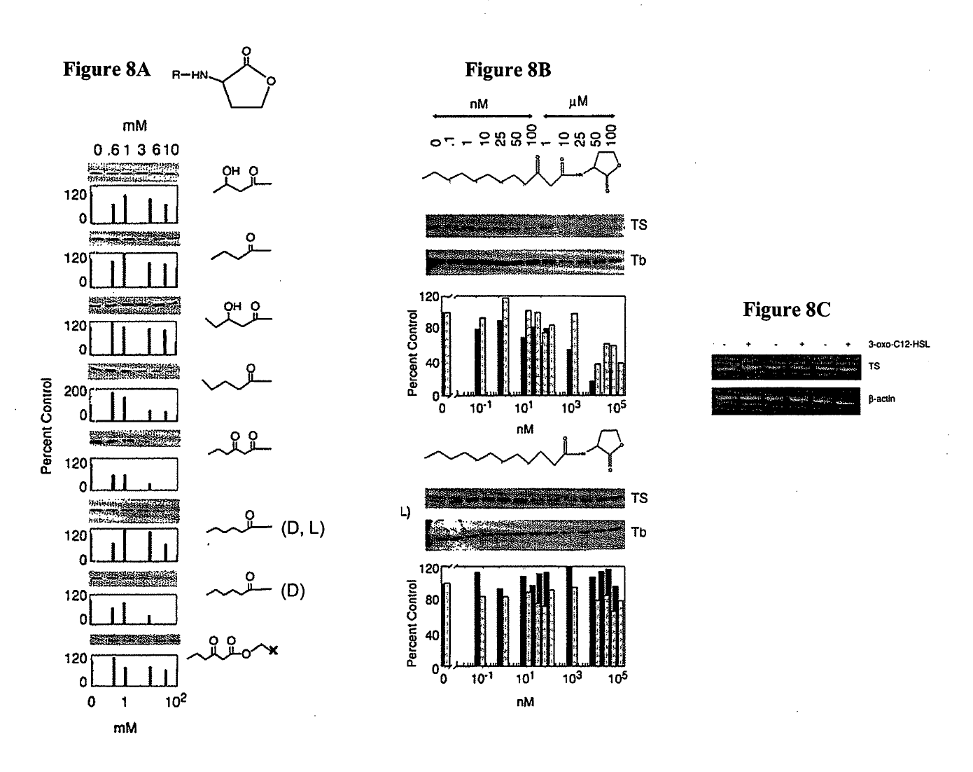

This Example describes the synthesis of AHLs. The structures of the compounds used in these Examples are presented schematically in FIG. 8A. An (L) or a (D, L) designation refers to the chiral α-carbon in the homoserine lactone ring. Specific AHL stereoisomers were prepared from their respective homoserine lactone precursors. AHLs are (L) stereoisomers unless otherwise specified. Preparation of representative AHLs was as follows.

N-hexanoyl (L-)-homoserine lactone were provided as follows. To 0.583 g (3 mmoles) of sodium caproate and 0.546 g (3 mmoles) of (S)-(−)-alpha-amino-gamma-butyrolactone hydrobromide in 25 mL dry acetonitrile (CH₃CN) were added 0.604 g (3.15 mmoles) of 1-[3-(dimethylamino)propyl]-3-ethylcarbodiimide hydrochloride. After stirring overnight, the CH₃CN was evaporated and the residue transferred to a separatory funnel with 100 mL of ethyl acetate (EtOAc). The EtOAc layer was washed with 50 mL of 10% ammonium chloride (NH₄Cl). The aqueous layer was extracted with 100 mL more of EtOAc and combined and dried (Na₂SO₄). The residue, after evaporation of the EtOAc, was chromatographed on a 2×39 cm column of silica gel eluting first with methylene chloride (CH₂Cl₂; 150 mL), then (4 acetone/20 CH₃CN/76 CH₂Cl₂) until the compound eluted. The combined fractions were evaporated, taken up in CH₂Cl₂, filtered, and concentrated to a minimum volume upon which a gel-like solid formed. The solid was collected and washed with petroleum ether and dried. TLC indicated one spot with R_{f} of 0.55 in (4 acetone/20 CH₃CN/76 EtOAc) using a lactone spray. The yield from four crops was 0.516 g or 40.3%.

Synthesis of 3-oxo-N-hexanoyl-L-homoserine lactones entails a) preparation of a protected 3-oxo-N-alkyl component and b) the coupling of its sodium salt to (L-)homoserine lactone. Preparation of the protected 3-oxo-N-hexanoyl component was performed as follows. Into a 250 mL round-bottomed flask were added 25 g (158 mmoles) of ethyl butyrylacetate, 9.69 mL (173.8 mmoles) of ethylene glycol, 60 mL of benzene, 0.025 g of p-toluenesulfonic acid and a few boiling chips. A Dean-Stark apparatus and condenser were attached and refluxing continued during an overnight period. (Salmi, E. J., Chem. Ber. 71, (1938), 1803-1808.).

Coupling of the protected 3-oxo-N-hexanoyl component with L-homoserine lactone was performed as follows. The ketal derivative (29.6 g) obtained above was saponified with one equivalent (73.2 mL of 2 M NaOH: 3 h, reflux), yielding 25 g (87%) of the ketal of sodium 3-oxo-N-hexanoate. This sodium ketal salt was coupled with (L-)homoserine lactone-HBr as in the preparation of N-acylhomoserine lactones described above. The ketone is liberated with dilute acid treatment and after chromatography and recrystallization, yielding 13.7 g (50.7%, overall) of 3-oxo-N-hexanoyl-(L-)homoserine lactone.

To synthesize phenylacyl homoserine lactones, 500 mg of PS-carbodiimide resin (Argonaut Technologies) (793.65 g/mol; 0.63 mmol; 4.0 mol equiv) was added to a 10 mL reactivial (Pierce). Phenylacylcarboxylic acid (1.7 mol equiv.) was dissolved in ˜3.5 mL dichloromethane and added to each reactivial. In a separate Erlenmeyer flask, γ-aminbutyrolactone HCl (1.0 mol equiv.) was dissolved by sonication in 2.5 mL ethanol, which was then divided equally among each reactivial (0.5 mL per reaction). The reactivial containing solution was irradiated by microwave (START system) for 1.5 min at 1000 W. Following reaction, the resin was removed by vacuum filtration through a medium frit into a 250 mL round-bottom flask. Solvents were removed in vacuo and the product was redissolved in a minimal volume of ethyl acetate by sonication. The final product was recrystallized by hexanes titration and confirmed by H-NMR and mass spectrometry.

To synthesize 3-oxo-phenylacyl homoserine lactones, 400 mg of PS-carbodiimide resin (Argonaut Technologies) (793.65 g/mol; 0.504 mmol; 2.0 mol equiv) was added to a 10 mL reactivial (Pierce). To the reactivial, phenylacylcarboxylic acid (1.0 mol equiv), 2,2-dimethyl-1,3-dioxane-4,6-dione (Meldrum's acid; 1.0 mol equiv), and 4,4-dimethylaminopyridine (1.05 mol equiv) were added with ˜5 mL dichloromethane. The reaction was allowed to stir overnight at room temperature. Upon completion, the resin was removed by filtration, and the solvents were evaporated. Product was redissolved in ethyl acetate and washed with 0.1N HCl. The organic layer was dried over magnesium sulfate, which was removed by filtration. The solvents were removed in vacuo and the final product was dissolved in ˜5 mL acetonitrile and transferred to a 10 mL reactivial (Pierce). To the reactivial, γ-aminobutryolactone HCl (1.0 mol equiv) and triethylamine (1.2 mol equiv) were added. The reaction mixture was then irradiated by microwave for 10 min at 100 W twice. Upon completion of microwave reaction, solvents were removed in vacuo. Crude product was redissolved in ethyl acetate with minimal water to aid solubility and washed once each with saturated Sodium bicarbonate, 1M Potassium bisulfate, and saturated Sodium Chloride. The organic layer was dried over magnesium sulfate, which was removed by filtration. Solvents were removed in vacuo and the resulting product was redissolved in a minimal volume of ethyl acetate and loaded onto a 20 g flash chromatography column. The final product was purified by flash chromatography (Flashmaster system; Argonaut Technologies) using the gradient system shown in Table 1.

TABLE 1

| Time | % Ethyl Acetate | Flow |
|---|---|---|
| 0 | 0 | 10 mL/min |
| 10 | 0 | 10 mL/min |
| 15 | 50 | 10 mL/min |
| 30 | 80 | 10 mL/min |
| 47 | 80 | 10 mL/min |

Fractions were collected and analyzed by thin layer chromatography (80:20 Ethyl acetate/hexanes). Fractions containing product were collected and solvents were removed in vacuo. The final product was confirmed by H-NMR and mass spectrometry (H-NMR results presented in Example 14).

### EXAMPLE 2

This Example demonstrates that rTS gene products can cause down regulation of TS expression. Conventional techniques were used to propagate and maintain the H630 and H630-1 cell lines (see, for example Dolnick, et al. (2003) Cancer Biology & Therapy, 2: 364-369; Stephanie, et al. (2001) In: Proceedings AACR, New Orleans, La., pp. 493). For the investigation of effects on TS expression, cells were extracted at 18-20 hours after the addition of AHLs. For Western blot analysis, proteins were extracted and Western blotting was performed by standard methods using blots blocked with 5% nonfat dried milk dissolved in 10 mM Tris-HCl, pH 8.0, 150 mM sodium chloride, 0.05% Tween-20. The blots were probed using either monoclonal (4D5E11) or polyclonal antibodies to TS and then probed for α-tubulin (monoclonal antibody B-5-1-2, Sigma Biochemicals) to confirm equivalent protein loading. The same blots were successively probed with anti-rTSβ or anti-TS, and finally anti-tubulin. After probing for each protein, the blots were stripped by incubation in 8 M guanidine HCl. Detection of rTS protein was performed with mouse monoclonal antibody D3, prepared against recombinant rTSβ protein. All Western blots were detected using the horseradish peroxidase-based West Pico Dura chemiluminescent substrate (Pierce) and horseradish peroxidase-coupled F(ab′)₂ fragments (Jackson ImmunoResearch Laboratories, West Grove, Pa.) as secondary antibodies

To illustrate that rTS gene products can cause down regulation of TS, human colon cancer cells (H630) and human colon cancer cells overexpressing rTSβ (H630-1) were designated as either donors or recipients based upon whether the cells were used as the source of diffusible materials or as recipients. TS was assayed in the recipient cells co-cultured with donor cells that were prevented from making contact with the donor cells through the use of a semi permeable membrane as a partition.

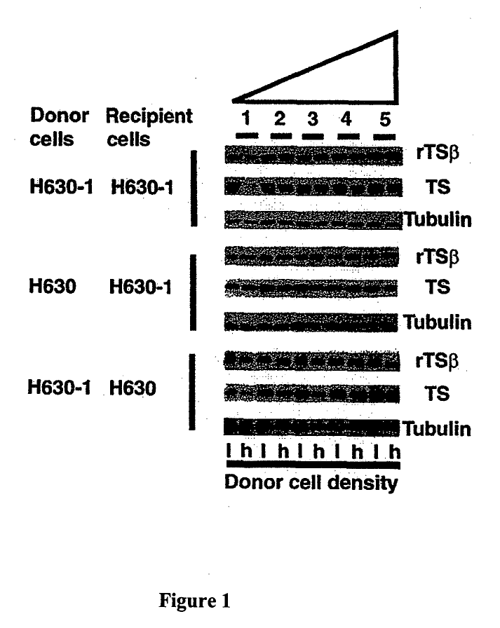

To obtain the data presented in FIG. 1, donor cells (H630-1 or H630) at low (“1”, 9.06×10⁴ cells/cm²) or high (“h”, 4.90×10⁵ cells/cm²) population densities were co incubated with recipient cells (H630-1 or H630) separated by semi permeable membranes (0.2μ) as follows. Donor cells were seeded at low or high densities in the wells of 6-well transwell plates. Recipient cells were seeded in duplicate at multiple densities in the inserts on separate dishes. Both donor and recipient cells were allowed to grow 4 days to reach the desired levels of confluence. Inserts containing recipient cells were then removed from their dishes, excess media was gently aspirated, and duplicate inserts transferred to donor wells containing low density (1) or high density (h) populations. The cells were incubated together overnight (18 hr) at 37° in a 5% CO₂/air humidified environment, then the inserts were removed. The insert cells were gently washed twice with cold phosphate buffered saline, extracted and analyzed by western blotting with antibodies to rTSβ, TS and tubulin. The results are shown in FIG. 1 for H630-1 donor and recipient cells; H630 donor cells and H630-1 recipient cells; and H630-1 donor cells and H630 recipient cells.

The data in FIG. 1 demonstrate that donor H630-1 cells at high population densities cause a decrease in the amount of TS protein in either recipient H630-1 or H630 cells, but that H630 cells do not cause a down-regulation of TS protein in H630-1 cells under the same conditions. The data indicate that low population density cells cannot cause a down-regulation in TS. Since the donor and recipient cell populations are not in direct physical contact, the decrease in TS protein must be caused by a diffusible substance, produced in abundance by H630-1 cells compared with H630 cells. FIG. 1 also shows that rTSβ protein levels increase with increasing cell population density in the recipient cells. Since neither tubulin or rTSβ are decreased in response to the donor cells, but TS is, this Example demonstrates that the H630-1 cells secrete a substance, or substances that cause a relatively specific decrease in the amounts of TS exposed to this substance or substances.

### EXAMPLE 3

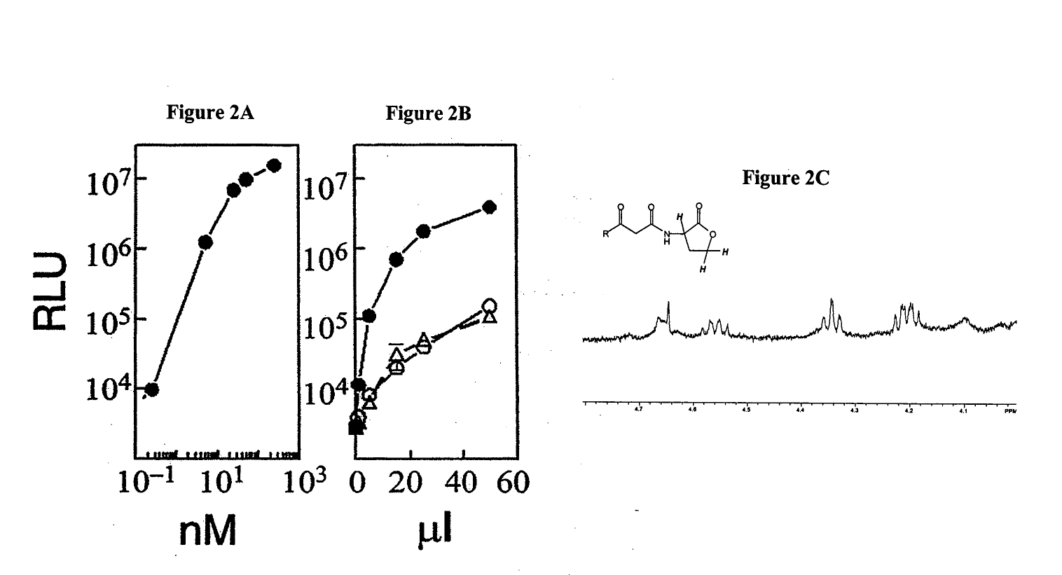

This Example demonstrates that conditioned medium from human cells contains AHLs. To illustrate this embodiment, extracts of media obtained from H630-1 cells, H630 cells or control culture medium were analyzed using a luciferase based bacterial bioassay (Winson et al., FEMS Microbiol. Lett, 1998, 163:193-202). Briefly, this assay makes use of a recombinant bacteria that does not normally make use of an AHL-based quorum sensing system The bacteria contains a recombinant plasmid that expresses luciferase protein under the control of an AHL receptor that was transfected into the bacteria. Exposure of the bacteria to AHLs, or other compounds that can bind to the receptor and induce the desired conformational changes, results in the expression of the luciferase protein and the production of luminescence. The measurement of luminescence reflects the extent of activation of the recombinant quorum sensing system. To perform the assay, cell culture medium (RPMI1640+10% dialyzed fetal bovine serum) was collected from tissue culture dishes containing either H630 or H630-1 cells grown to 80-95% confluence, or from control medium (stock solutions without cells). The medium (500 ml) was acidified and extracted three times with methylene chloride. The extracts were dried with an excess of magnesium sulfate and the filtered material evaporated to dryness and redissolved with 500 μl of 10% dimethyl sulfoxide. The indicated amounts of collected material or solvent were assayed in duplicate for their ability to stimulate luminescence in a bioassay. The results are shown in FIGS. 2A and 2B. FIG. 2A shows the results obtained with 3-(oxo)-hexanoyl-homoserine lactone which is the natural signaling molecule in bacteria for the recombinant receptor used in the assay. The relative light units (RLU) produced are an indication of the amount of molecules present in a sample of H630-1 media extract that can activate the receptor(●), H630 media extract (◯) and RPMI1640+10% dialyzed fetal bovine serum control media extract (Δ) in FIG. 2B. The results show increased response (approximately 40-fold) with the extracts prepared from the rTS over expressing cells (H630-1) compared with the non-over expressing cells (H630) and the control medium. The increase in response with the rTS overproducing cell line is directly proportional to the increased production of rTSβ protein by the H630-1 cell line.

The presence of AHLs in the culture media was established by separating extracted media using thin layer chromatography and analyzing one of the eluted spots using ¹H-NMR for the presence of homoserine lactone ring protons. The results from this analysis are presented in FIG. 2C. The top spectrum is an analysis of the H630-1 extract. The bottom spectrum is an analysis of a synthetic acyl homoserine lactone with the ring protons labeled as shown (H). The three peaks observed between 4.1 and 4.6 ppm represent the three ring protons in the structure. The patterns are nearly identical, indicating the production of an AHL molecule by the H630-1 cells. Thus, this Example confirms that H630-1 cells secrete an AHL into the conditioned medium.

### EXAMPLE 4

This Example demonstrates that H630-1 cells produce AHL molecules using a biochemical pathway similar to that found in bacteria. In bacteria, the AHL biosynthetic pathway makes use of fatty acids to provide the acyl side chain and S-adenosylmethionine to provide the homoserine lactone ring (More, et al., 1996, Science 272, 1655-1658). The carboxyl carbon of methionine is incorporated into AHLs whereas the methyl carbon and sulfur of methionine are not. Consequently [¹⁴COOH]-methionine (after conversion to [¹⁴COOH]—S-adenosylmethionine) will lead to the production of acyl-[¹⁴C]-homoserine lactones, whereas [14CH₃]-methionine and [³⁵S]-methionine will not. All three radiolabelled atoms in methionine are incorporated into proteins and nucleic acids. The hydrophobic properties of the lactone ring and the acyl side chains enable AHLs to be selectively extracted into organic solvents at acid pH, compared with peptides or DNA or RNA. This strategy, which allows for the purification of AHLs from bacterial media, was used to characterize substances in mammalian culture media.

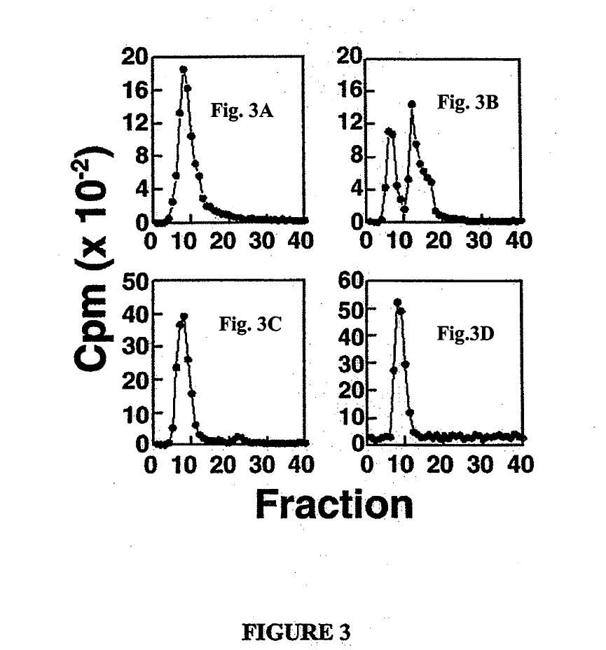

To confirm that H630-1 cells were synthesizing the AHL signal molecules using a biochemical pathway similar to that found in bacteria, H630 and H630-1 cell lines were grown in the presence of radiolabeled methionine as follows. H630-1 or H630 cells grown in RPMI 1640 medium containing 10 μM methionine and 10% dialyzed fetal bovine serum were grown in 25 cm² culture flasks until approximately 70% confluent. The medium was aspirated and the cells gently washed with medium lacking methionine. Fresh medium containing 10 μM methionine supplemented with 10% dialyzed fetal bovine serum and 25 μCi radiolabelled methionine (56 mCi/mmol [¹⁴COOH]—, 55 mCi/mmol [¹⁴CH₃]—, or 1175 Ci/mmol [³⁵S]-methionine) was added to a concentration of 5 μCi/ml and the cells incubated at 37° C. for 16-18 hr. The medium was collected and filtered (0.2μ) to remove detached cells and debris, then acidified with HCl and extracted five times with ethyl acetate (2.4 volumes). Upon alkalinization, greater than 95% of the radioactivity was found to remain in the aqueous phase. The organic extract was dried with MgSO₄ and the ethyl acetate removed under vacuum at 40° C. The residue was dissolved with 50% methanol and sequentially filtered through Centriprep filters (Millipore) with 10,000 and 3,000 MWCO, respectively to reduce contamination by peptides and oligonucleotides. The sample was then concentrated in a speed-vac and redissolved with 0.1 original volumes of acetonitrile. The column used was a Luna C18(2) 4.6×250 mm, 5 μm (Phenomenex). Prior to HPLC, the acetonitrile was removed and the sample was dissolved in the starting buffer (90:10:0.1 H₂O:CH₃CN:trifluoroacetic acid). After loading the sample the column was developed with a linear gradient of starting buffer containing 10-80% CH₃CN at 0.5 ml/min. The amount of material loaded in FIG. 3 was approximately 10⁴ cpm (A, B) or 2×10⁴ cpm (C, D) and 0.5 ml fractions were collected and assayed for radioactivity.

The elution profile of AHLs in this system is dependent upon the length, oxidation state, and degree and types of substitution and saturation of the acyl side chain, with shorter and more oxidized species eluting earlier. As illustrated in FIG. 3, [¹⁴COOH]-methionine gives rise to unique ethyl-acetate extractable material present in fractions 12-17 from H630-1 spent medium compared to the extract with spent medium obtained from H630 cells (compare FIGS. 3A and 3B). The absence of [¹⁴C]— and [³⁵S]-methionine metabolites in fractions 12-17 after labeling cells with [¹⁴CH₃]— (FIG. 3C) or [³⁵S]-methionine (FIG. 3D) argues against the possibility that the novel peaks observed in FIG. 3B arise from hydrophobic peptides or RNA fragments. The elution times of the H630-1 generated [¹⁴COOH]— metabolites are characteristic of AHLs with acyl side chains having four or more carbons (e.g., six carbon chains) and containing polar substituents such as hydroxyl or keto groups (Camara et al., 1998, Meth. Microbiol. 27, 319-330. Fractions 12-17 appear to contain a heterogeneous mix of metabolites with similar elution properties.

These data demonstrate that the only radiolabelled atom from either ¹⁴C—, or ³⁵S— labelled methionine that is incorporated into a substance present in the H630-1 cell culture medium (present in the second peak contained in fractions 12-16), that can be extracted with an organic solvent such as ethyl acetate, is the carbon of the carboxyl group on methionine. The results also demonstrate a high level of secretion of methionine metabolites from H630-1 cells (FIG. 3B), compared to H630 cells (FIG. 3A) that contain the ¹⁴C derived from ¹⁴COOH, but that do not contain the ¹⁴C from the methyl (CH₃) of methionine or the ³⁵S from the sulfur in methionine. While methionine can be metabolized extensively in the mammalian cell such that the radiolabelled atoms can be incorporated into substances such as protein and RNA, the appearance of fractions uniquely labeled with ¹⁴C—[COOH]-methionine from the rTS-overproducing cells (H630-1), but none of the other radiolabelled sites, or H630 cells, demonstrates that the material being extracted contains the portion of methionine used to synthesize a homoserine lactone ring by a biosynthetic pathway similar to the bacterial biosynthetic route for acyl-homoserine lactones (Dolnick et al, 2003, Cancer Biol. & Ther. 2:364-369). Thus, this Example, in conjunction with Example 4, demonstrates that H630-1 cells synthesize AHLs, and that the synthesis is via a biochemical pathway similar to that found in bacteria cells.

### EXAMPLE 5

This Example demonstrates the involvement of the rTS gene product in the synthesis of acyl homoserine lactones. To illustrate this, synthesis of acyl-¹⁴C-acyl-homoserine lactones by purified rTSβ protein was studied. Plasmid pET24 a (+) containing cDNAs for either rTSα or rTSβ were transfected into the Escherichia coli strain BL21[DE3] and induced to express the rTS proteins by the addition of the inducing agent IPTG (isopropyl-β-thiogalactoside). The proteins, expressed as insoluble inclusion bodies were purified by differential extraction and refolded using the non-ionic sulfo-betaine NDSB 201 as follows.

Solubilized inclusion bodies containing 2-4 mg/ml rTS proteins were heated at 70° C. for 15 min to disrupt aggregates then adjusted to 2 mg/ml and incubated on ice for 1 hr. The samples were centrifuged (100,000×g×10 min) and the supernatant used immediately for refolding. All the following operations were conducted at 4° C. The supernatants containing solubilized protein were added to refolding solutions consisting of 1× Foldase III buffer (GenoTech) supplemented with 5 mM DTT. The refolding solution was stirred rapidly during addition of the solubilized proteins and stirring was continued for 1 h. The resulting solutions were then dialyzed against 50 mM TRIS-HCl pH 8.0, 300 mM NaCl, 0.1 mM EDTA and 7.5% glycerol with one change of buffer. The dialyzed protein solutions were quantitated spectrophotometrically. Samples were stored at −80°. Upon thawing, bovine serum albumin was added to 1 mg/ml and the proteins were concentrated with a microcon 30 (Millipore) 6-fold (adjusting the final volume as needed).

The enzyme assay was performed using standard techniques and essentially as previously described (More et al., 1996, Science, 272:1655-1658). Briefly, this assay is based upon the premise that rTS will synthesize acyl homoserine lactones from S-adenosylmethionine and a fatty acid donor (e.g. fatty acyl-protein), as bacteria do. Since the fatty acyl portion of the AHLs produced by humans is undefined, the assay designed to provide one defined substrate for AHL synthesis (¹⁴COOH—S-adenosylmethionine) to assay synthesis of an AHL. The fatty acid substrate for the synthesis is provided by a crude extract (i.e., the S28 extract) prepared from bacteria known not to make acyl-homoserine lactones. Since bacteria make fatty acyl-proteins, they provide a heterogeneous source of these materials. Only fatty compounds to which the ¹⁴COOH of S-adenosylmethione is transferred will be extracted into organic solvent after an enzyme catalyzed reaction. As a result, the synthesis of acyl-homoserine lactones can be evaluated even without knowing the doner for the acyl portion of the enzyme product. The assay utilizes refolded rTS protein, or dialysis buffer processed as for the refolded proteins, combined in duplicate reactions on ice with a reaction cocktail set up to contain in a final volume of 100 μl the following components: The indicated amounts of rTS protein or equal amounts of dialysis buffer containing bovine serum albumin solution (as a control), 9 mM MgSO₄, 1 mM DTT, 297 mM NaCl, 90 μM FeSO₄, 45 mM TRIS-HCl pH 7.5, 45 μM [¹⁴COOH]—S-adenosylmethionine (0.135 μCi), 896 μM ATP, 0.9 mM NADPH, and 11.4 mg/ml S28 extract (More, et al. (1996) Science, 272: 1655-1658). The reactions were incubated at room temperature for 2 hr and stopped by the addition of 20 μl 10% trichloroacetic acid. Ethyl acetate was then added (200 μl) and the samples vortexed well. The organic and aqueous phases were separated by centrifugation and the organic layer was removed and radioactivity measured. Controls and standards consisted of reactions run in the absence of rTS proteins and mock reactions counted in their entirety to provide a reference for total radioactivity per reaction. Product formation (pmol) was calculated based upon the net fraction of input radioactivity extracted into the organic phase.

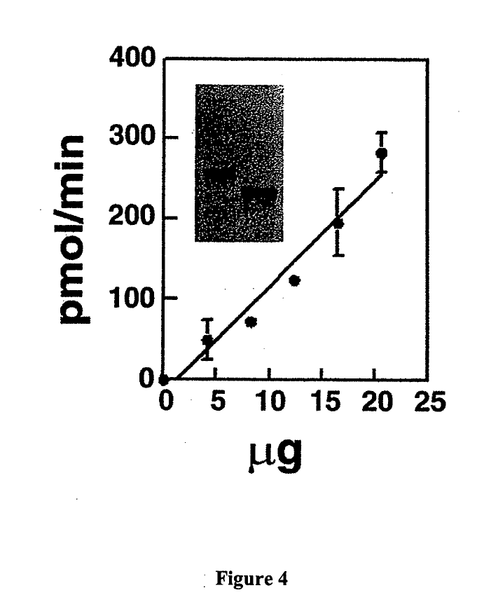

The results are shown in FIG. 4. The inset shows the Coomassie blue staining pattern for the two proteins (rTSβ, left; rTSα, right) separated on denaturing polyacrylamide gels and demonstrates that the proteins are pure. No activity was found to be associated with rTSα protein, so only results for rTSβ protein are presented. The increased incorporation of ¹⁴C—[COOH]-methionine into an ethyl acetate extractable material, proportional to the amount of rTSβ protein added to the reaction demonstrates a direct role of rTSβ in synthesizing acyl-homoserine lactones. Although there may be other roles for the rTSβ protein, these results suggest that the rTSβ protein acts as an AHL synthase.

### EXAMPLE 6

This Example provides an analysis of the effects on TS of various AHLs with altered lengths. In particular, the effect of AHLs with four carbon (C4) or six carbon (C6) length acyl side chains was tested on H630 cells.

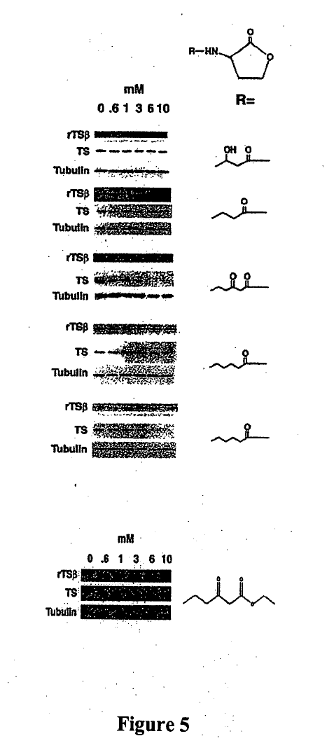

As shown in FIG. 5, the indicated AHLs (top to bottom: 3-hydroxy-butanoyl homoserine lactone, butanoyl homoserine lactone, 3-(oxo)-hexanoyl homoserine lactone, hexanoyl-homoserine lactone, hexanoyl-(L)-homoserine lactone) were used. These AHLs were synthesized as described previously (Chhabra, et al., 1993, J. Antibiotics 46, 441-454). For studies with the H630-1 cell line, AHLs were either dissolved in RPMI 1640 (hydroxy-butanoyl-, butanoyl-, 3-(oxo)-hexanoyl-homoserine lactones) or in dimethyl sulfoxide (DMSO) (hexanoyl-homoserine lactones) and added to mid-log H630 cells in RPMI1640 containing 10 μM methionine and 1× ITS+ culture supplement (BD Bioscience) in place of serum at the indicated concentrations. For AHLs dissolved in DMSO, parallel cultures were supplied with equivalent amounts of DMSO to control for solvent effects, and were found to be unaffected. Because medium supplemented with serum showed evidence of a quorum sensing bioactive material (FIG. 2), a serum substitute was employed. After 18 hr, the medium was removed, the cells washed twice and protein extracted. The extracted proteins were separated on denaturing polyacrylamide gels and probed for rTS, TS and tubulin using specific antibodies.

The results are shown in FIG. 5. Western blots (left) show TS is down-regulated by some AHLs over the indicated concentration range. The levels of rTSβ and tubulin were unchanged. The compound 3-(oxo)-ethylhexanoate was tested for activity, as control for 3-(oxo)-hexanoyl homoserine lactone, and was found to be inactive (data not shown). These results show that the six carbon (C6) AHLs are biologically active but the four carbon (C4) AHLs are not. Thus, this embodiment demonstrates that exogenous AHLs can down regulate TS.

### EXAMPLE 7

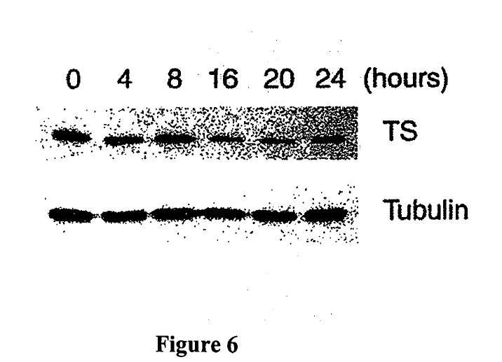

This embodiment describes the down regulation of TS by AHLs as a ftmction of time. H630 cells were incubated with medium containing 3-oxo-hexanoyl-L-HSL (3 mM) as described for Example 5. The extraction and analysis was also carried out as described in Example 5 at the indicated times after drug addition. The results are shown in FIG. 6. As can be seen, the same amount of 3-oxo-hexanoyl-homoserine lactone that reduced TS levels by approximately 50% (FIG. 5) after 16 hours, induces this effect between 8 and 16 hours after addition and maintains the effect for approximately 24 hours.

### EXAMPLE 8

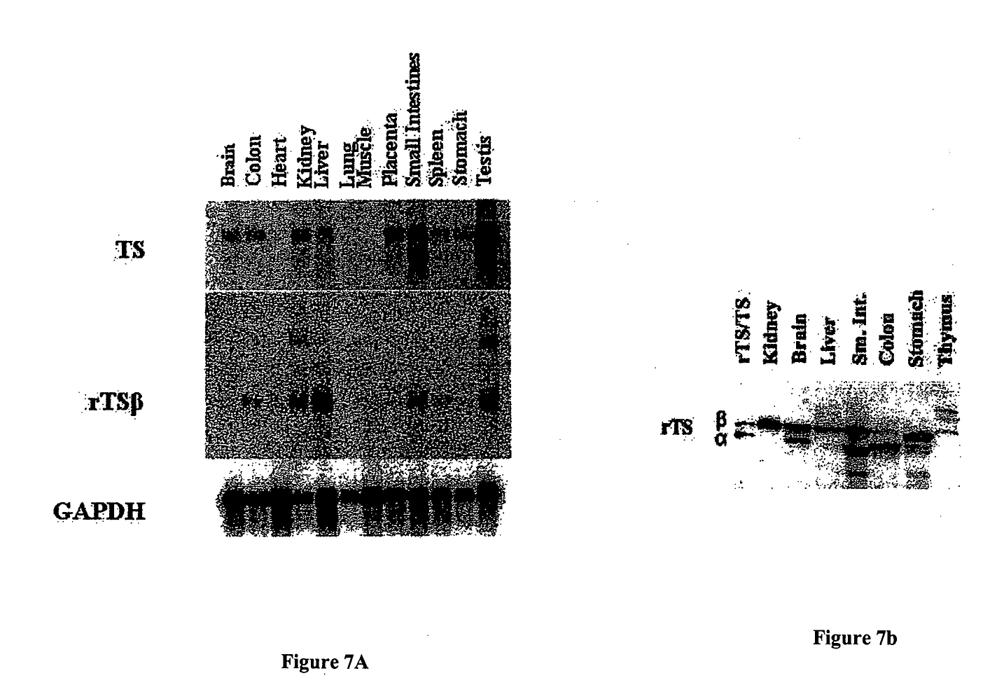

This embodiment demonstrates that the products of the rTS gene are present in a variety of normal tissues. To demonstrate this, rTSα and β proteins were detected using conventional immunoblot methods (FIGS. 7A and 7B). Both rTSα and rTSβ proteins are detectable in multiple organs as indicated. The lane on the left of FIG. 7A shows recombinant rTS proteins with a HIS6 tag used as internal markers. The organ proteins used for these studies were obtained from CloneTech as individual or pooled samples from deceased trauma victims. For the analysis provided in FIG. 7B, rTSβ, TS (TS) and glyceraldehyde phosphate dehydrogenase (GAPDH) mRNAs were detected by conventional methods using a poly RNA blot obtained from Origene. The lack of agreement in the expression of rTS proteins compared with mRNAs (for example in brain and stomach) demonstrates the variability in expression of the rTS gene between samples.

### EXAMPLE 9

This Example demonstrates that an AHL of the invention is effective at low concentrations in down-regulation of the expression of TS protein.

A variety of AHLs with acyl-chains containing 4- to 12-carbons (C4-C12) and 3-hydroxy or 3-keto substituents were prepared as described in Example 1 and analyzed for their effect on TS protein expression by Western blotting. The results are presented in FIG. 8A and FIG. 8B. Although several of the shorter chain AHLs (C4-C6) can cause loss of TS, the required concentrations are in the mM range. The (L)-isomer of C6-HSL is active whereas the racemic mix is less active, indicating that C6-(D)—HSL is inactive for down-regulation of TS. In contrast to the short chain AHLs, 3-oxo-C12-HSL can cause down-regulation of TS at concentrations as low as 10 μM (FIG. 8B), while C12-HSL, which lacks the 3-oxo group, is inactive at concentrations as high as 100 μM. The observed difference in activities when a 3-oxo group is present (3-oxo-C12-HSL versus C12-HSL) suggest that the down-regulation of TS by 3-oxo-C12-HSL is receptor mediated. Tubulin, a protein frequently employed as an internal standard for protein loading also appears to decrease with increasing concentrations of 3-oxo-C12-HSL, but not with increasing concentrations of C12-HSL (FIG. 8B). The latter observation indicates 3-oxo-C12-HSL has effects on gene expression aside from the down-regulation of TS. In this regard, FIG. 8C provides a photographic representation of the effect of 23 μmol/L 3-oxo-C12-HSL on TS mRNA levels in H630 cells (20 hours of exposure) as evaluated by RT-PCR. For these experiments, TS mRNA expression was evaluated in 3-oxo-C12-HSL treated cells by reverse transcription-PCR (RT-PCR) using standard techniques and as previously described (Wu et al. (2003) Mol. Pharmacol; 63:167-73.). Microarray analysis of H630 RNA was done using the Affymetrix (Santa Clara, Calif.) HGU133A GeneChip. Microarray data was analyzed using Genetraffic software (Stratagene, La Jolla, Calif.) with the Robust Multichip Analysis method (Lauren et al., (2003) IEEE Trans Nanobioscience; 2:163-70) for measuring probe intensity and scaling. Results for treated and untreated cells were analyzed for significant changes within each experiment (P<0.05) as well as for changes in pooled data from three separate experiments. RNA for RT-PCR or microarray analysis was prepared using an RNAeasy kit from Qiagen (Valencia, Calif.). Each analysis made use of RNA from control cells and cells treated 20 hours with 23 μmol/L 3-oxo-C12-HSL prepared on three separate occasions from H630 cells. As shown in FIG. 8C, TS MRNA levels are unaffected by 3-oxo-C12-HSL and also failed to identify a single gene that is significantly altered in response to treatment with the compound (data not shown). These results indicate the effects of 3-oxo-C12-HSL on gene expression 20 hours after treatment of cells are due to post-translational events. Thus, this Example demonstrates that 3-oxo-C12-HSL can downregulate TS protein expression at low concentrations and does not appear to adversely or otherwise affect the expression of mRNAs for genes other than TS.

### EXAMPLE 10

This Example demonstrates the effect of particular AHLs on the translational autoregulation of TS mRNA. Also demonstrated are the cell growth inhibitory effects of AHLs on (in addition to H630 cells) MCF-7 cells, a breast cancer cell line, and NCI-H460, a lung cancer cell line.

Cells used in the growth inhibition experiments were routinely screened and found to be negative for mycoplasma. Growth inhibition and colony formation assays were performed using six well tissue culture dishes. For growth inhibition studies, wells of 6 well tissue culture plates were plated in triplicate with 10⁵ cells in 2 ml of medium and the compounds to be tested were added after 24 hours as 100- or 1000-fold concentrated stock solutions. Compounds were dissolved in growth medium or for the C12 compounds in dimethyl sulfoxide (0.1% final concentration). For studies involving the C12 compounds, all wells were adjusted to contain 0.1 % dimethyl sulfoxide. For growth inhibition studies, the cells were counted 3 days after the addition of AHLs and the data for treated cells normalized as described below. The medium was aspirated and the cells were carefully rinsed twice with ice-cold PBS (137.9 mM NaCl, 2.7 mM KCl, 1.5 mM KH₂PO₄, and 6.5 mM Na₂HPO₄) then harvested by trypsinization. Cell numbers were determined using a hemacytometer For colony formation assays, cells (200 and 500 cells/well for H630 and H630-1, respectively) were plated in triplicate. The compounds of interest were immediately added, then the cells were allowed to grow for approximately 2 weeks (until colonies were visible by eye). The colonies were stained with crystal violet and counted. Resulting values for both the growth inhibition assays of this Example and colony formation assays (Example 11) are expressed as percent control cell growth or control colony number. For colony formation assays involving two compounds, the AHL was present at the concentration needed for 50% growth inhibition after 3 days (IC50, 23 μM for H630 and 8 μM for H630-1). For graphic presentation, the values for cells treated with two compounds were normalized by multiplying these values by a number that gives 100% for cells treated with AHL alone. Data presented for cells treated with one compound in both types of growth assays are normalized to either untreated cells or cells to which solvent was added. All results are expressed ± the standard deviation. All experiments were repeated at least twice with similar results. Also provided in this Example is an analysis of a long chain (3-oxo-C12-HSL) and a short chain (3-oxo-C6-HSL) AHL for their ability to modulate TS in a TS modulation assay (Wu, et al. (2003) Mol Pharmacol, 63: 167-173). Briefly, this assay relies upon the ability of TS protein to bind to TS binding elements (TBE) present in certain mRNAs. When TS binds to mRNAs containing TBEs, the outcome is known to be inhibition of mRNA translation, and an inhibition of new protein synthesis. In the TS modulation assay, a recombinant plasmid was constructed with the luciferase mRNA containing TBEs. Cells were prepared expressing this plasmid such that the luciferase protein is not expressed in the cells when TS is present. Any compound that causes TS to be removed from the recombinant luciferase mRNA causes luciferase to be expressed. The luciferase expression is then quantified by measuring luminescence. Compounds that are known to be active either inhibit or bind to TS (such as 5-fluorouracil or tomudex). Since compounds that cause the down-regulation of TS will have the same effect on the mRNA because there will not be enough TS to bind to the recombinant luciferase mRNA, these will also cause up regulation of luciferase (Wu, et al. (2003) Mol Pharmacol, 63: 167-173; Chu, et al. (1996) Bioessays, 18: 191-198). To perform this assay, H630 cells were treated with IC50 concentrations of each compound.

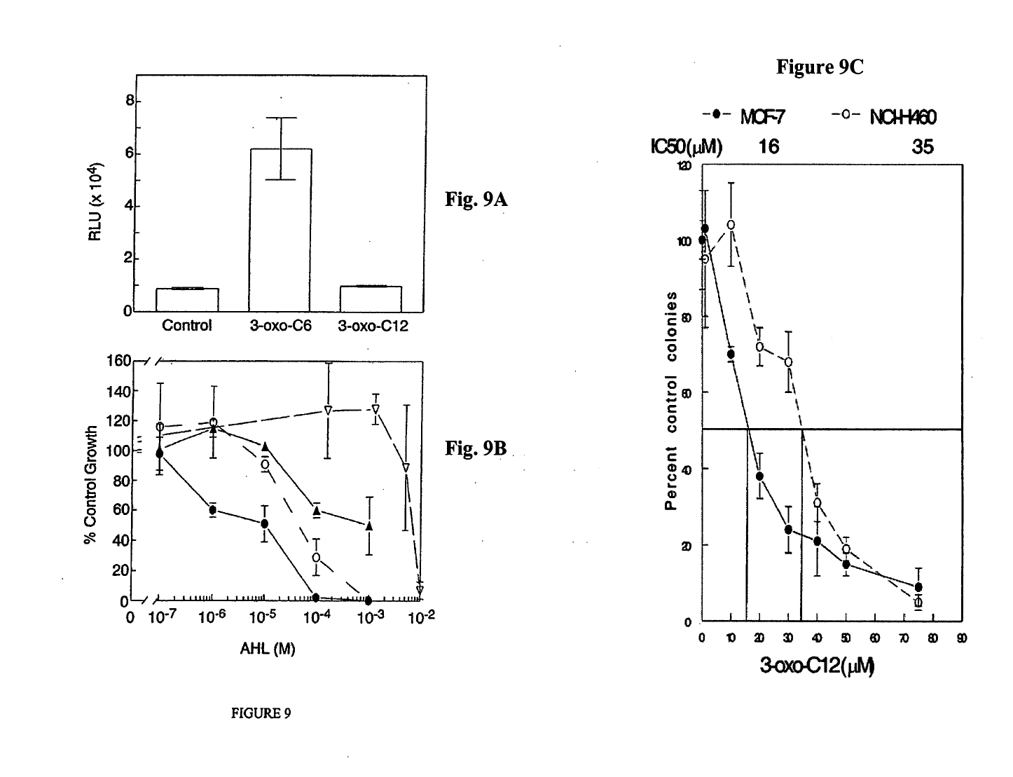

The results from the TS modulation assay are presented in FIG. 9A. The results from the cell growth inhibition assays are presented in FIGS. 9B and 9C. As can be seen in FIG. 9, The shorter chain compound 3-oxo-C6-HSL is a potent modulator of TS, much more so than the more classical TS inhibitors studied previously using this assay (Wu, et al. (2003) Mol Pharmacol, 63: 167-173). In contrast, 3-oxo-C12-HSL, though a much more effective down-modulator of TS (FIG. 8), has no detectable TS modulatory activity in this assay. Despite the lack of activity of 3-oxo-C12-HSL in this TS modulation assay, the concentration dependence of H630 growth inhibition by this compound closely mimics the down-regulation of TS (compare FIG. 8B with 9B, where 8B shows the concentration of 3-oxo-C12-HSL needed to reduce the amount of TS in H630 cells is approximately 1-10 μM compared with an IC50 of approximately 10 μM for inhibition of cell growth in a clonogenic assay [solid circles in 9B]). For FIG. 9B: Solid circles (•), 3-oxo-C12-HSL; Open circles (∘), 3-oxo-C12-(D,L)-HSL; Closed triangles (▴), C12-HSL; Open triangles (∇), 3-oxo-C6-HSL.

Unlike 3-oxo-C6-HSL, the growth inhibition curve for 3-oxo-C12-HSL appears biphasic, indicating there may be a second site of action which may interfere with the modulation assay, providing further support for the existence of multiple AHL receptors. Thus, these data indicate that TS is an important, but may not be the only target for the growth inhibitory effects of 3-oxo-C12-HSL, and observation further supported by the down-regulation by 3-oxo-C12-HSL of tubulin, a microtubule protein (FIG. 8b).

FIG. 9B shows growth inhibition data for the 3-oxo-(D,L)—C12-HSL and C12-HSL. It is evident that, like C6-HSL, the (L)-stereoisomer of 3-oxo-C12-HSL is more active than the racemic mixture. FIG. 9B also shows that C12-HSL (which lacks the 3-oxo moiety) is a relatively poor growth inhibitor (IC50=1 mM), consistent with its lack of effect on TS. However, as depicted in FIG. 9C, 3-oxo-C12-HSL is also effective at inhibiting the growth of MCF-7 cells, a breast cancer cell line, and NCI—H460, a lung cancer cell line. The IC50 values are 16 μM for MCF-7 and 35 μM r for NCI—H460. Therefore, this Example demonstrates that the growth of several distinct cancer cell types can be inhibited using AHLs.

### EXAMPLE 11

This Example demonstrates that 3-oxo-C12-HSL can enhance the cell growth inhibitory activity of 5-fluorouracil (FU).

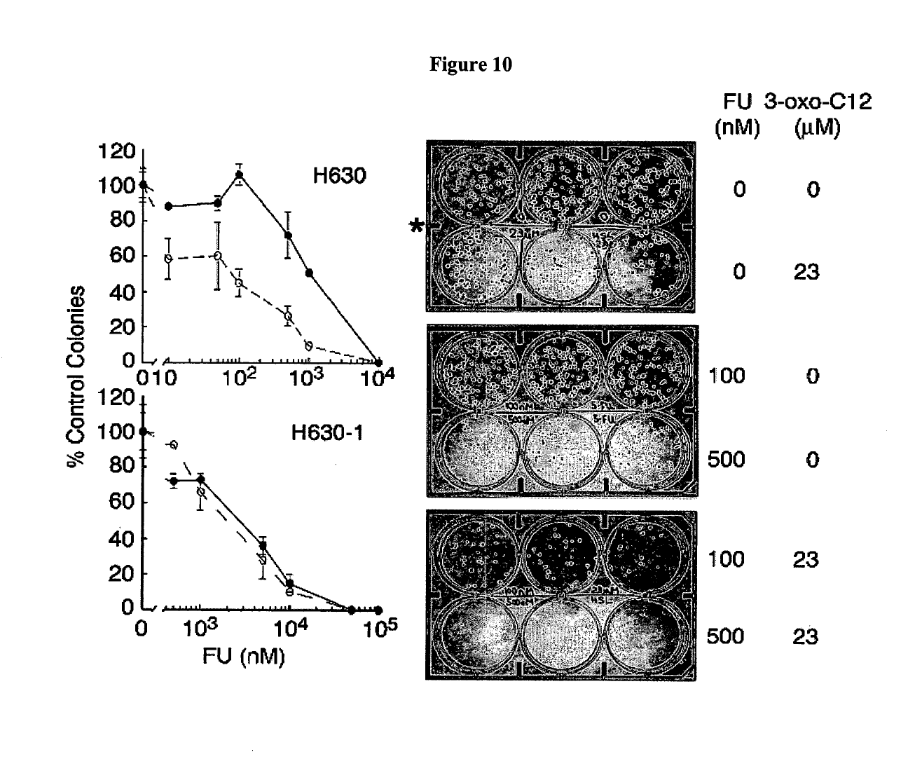

To analyze whether the down-regulation of TS by AHLs may have therapeutic utility, either H630 or H630-1 cells were allowed to form colonies in the presence of increasing concentrations of FU, either with FU alone, or in combination with an IC50 amount of 3-oxo-C12-HSL. The results are depicted in the graphs of FIG. 10 (presence of FU alone: (•); FU in combination with 3-oxo-C12-HSL: (∘)). The results indicate there is about a 12-fold enhancement (80 vs 1000 nM) in the apparent IC50 of FU in H630 cells in the presence of 23 μM 3-oxo-C12-HSL. No enhancement is observed with H630-1 cells, indicating either that the elevated expression of TS or other genetic or epigenetic changes in the FU-resistant cells preclude an effect. For the H630 cells, there is a shift of the inhibition curve to the left in the presence of 3-oxo-C12-HSL. A sample photograph of results for a subset of drug concentrations is shown on the right. Thus, 3-oxo-C12-HSL can enhance the growth inhibition effect of FU on cancer cells because in a synergistic manner becase, if the results were merely additive, the inhibition of colony formation resulting from combining two drugs at their IC50 values would be a reduction in colony number of 75%. However, under the conditions used for the assays depicted in FIG. 10, the inhibition of colony formation resulting from combining IC50 amounts of 3-oxo-C12-HSL and FU is more than 90%, which is greater than additive.

### EXAMPLE 12

This Example provides further demonstartion of the enhancement effect of 3-oxo-C12-HSL on chemotherapeutic agents. The analysis was expanded to include colony formation assays using prodrugs that predominantly target TS (such as FUdR and tomudex), a compound that is metabolized to both a TS inhibitor and to an RNA precursor (FUrd), a drug that targets dihydrofolate reductase (methotrexate) and agents generally considered to not have an effect on TS (taxol and adriamycin). For these assays, 3-oxo-C12-HSL was present at the concentration needed for 50% growth inhibition after 3 days (IC50, 23 μM for H630 and 8 μM for H630-1). For graphic presentation, results for cells treated with two compounds were normalized by multiplying the result by a number that gives 100% for cells treated with AHL alone.

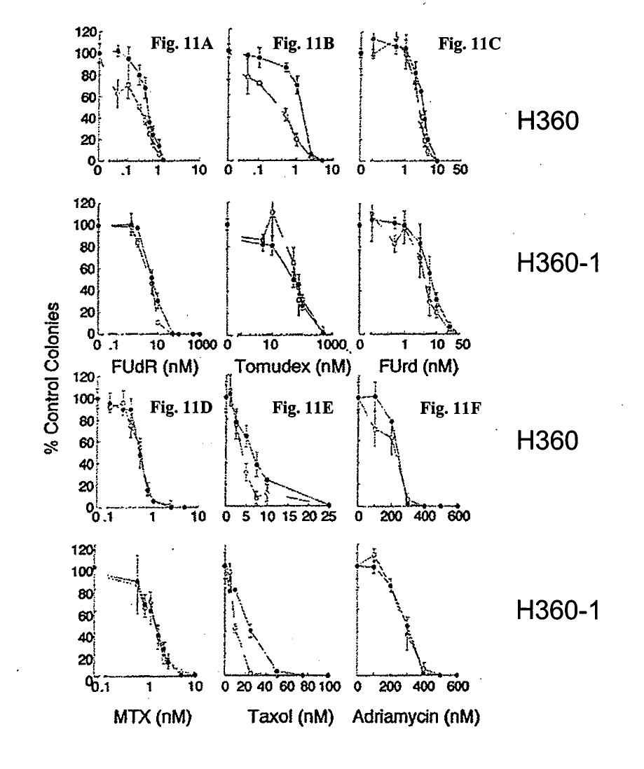

The results are presented in FIG. 11A-F for both H630 (top panels) and H630-1 cell lines (bottom panels), with test compound alone (•) or in combination with 3-oxo-C12-HSL (∘). The results are consistent with 3-oxo-C12-HSL acting on TS. The curves for FUdR and tomudex with H630 cells (FIGS. 11A and 11B) both show enhancement of the activity of the compounds. In contrast, no enhancement of FUrd occurs (FIG. 11C).

Enhancement of the effect of FU, FUdR and tomudex, but not with 5-fluorouridine (FUrd), methotrexate, or adriamycin, indicates that the effects of 3-oxo-C12-HSL upon TS are a major site of its growth inhibitory activity. It was therefore surprising to see some enhancement of the effect of taxol (FIG. 11E). Unlike the interaction of 3-oxo-C12-HSL with the TS inhibitors, some enhancement of the activity of taxol is seen with both cell lines. The data in FIG. 11E also show that the H630-1 cells are less sensitive to taxol (IC50=20 nM) than the H630 (IC50=6 nM) cell line. Thus, this Example demonstrates that 3-oxo-C12-HSL can enhance the effect of compounds known to predominantly target TS, as well as the effects of taxol on FU sensitive (H630) and insensitive (H630-1) cells.

### EXAMPLE 13

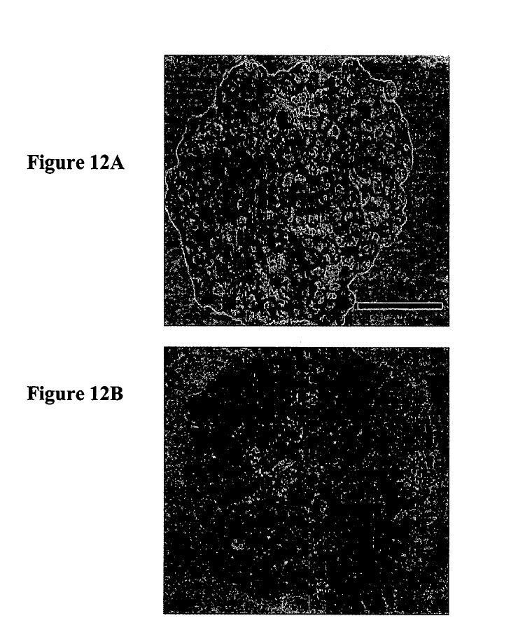

The Example provides an analysis of the alterations in the response of H630-1 cells to the microtubule inhibitor taxol by investigating alterations of microtubule-related proteins and microtubule function in the H630-1 cell line. Western blot data for AHL effects on H630 cells indicates that α-tubulin expression is affected by 3-oxo-C12-HSL (FIG. 8B). A 2D microarray analysis of the NCI 60 cell line panel for genes whose expression correlates with rTSβ indicates that both tubulin (R=−0.57, P<1.78e⁻⁶) and β-actin (R=−0.61, P<3.38e⁻⁷) are among the ten genes whose expression is most closely negatively correlated with rTSβ expression. Both tubulin and β-actin are associated with microtubule function and the cytoskeleton, which is responsible for maintenance of overall cell shape and which plays an important role in cell division and motility. Since H630-1 cells overexpress rTSβ 10- to 40-fold and 3-oxo-C12-HSL, but not C12-HSL, appears to have an effect on tubulin expression (FIG. 8B), overproduction of rTS signaling molecules may have effects on microtubule function and the cytoskeleton. We have demonstrated that the two cell lines are morphologically distinct, and that methionine restriction caused H630 cells to more closely resemble H630-1 cells (Stephanie, et al. (2001) In: Proceedings AACR, New Orleans, La., pp. 493). This difference is consistent with changes in the cytoskeletal makeup between the cells, as well as a connection between methionine metabolism and the cytoskeleton, and is evidenced by examination of a photomicrograph comparing H630 and H630-1 cells, (FIGS. 12A and B). It is apparent that H630-1 cells are more squamous, have pseudopodia and some cells with multiple nucleoli, all evidence of an altered cytoskeletal makeup.

### EXAMPLE 14

This Example demonstrates the effectiveness in inhibiting cancer cell growth of AHLs having an aromatic group at the end of the acyl chain, and that these AHLs are effective in inhibiting the growth of prostate cancer cells, in addition to colorectal cancer cells.

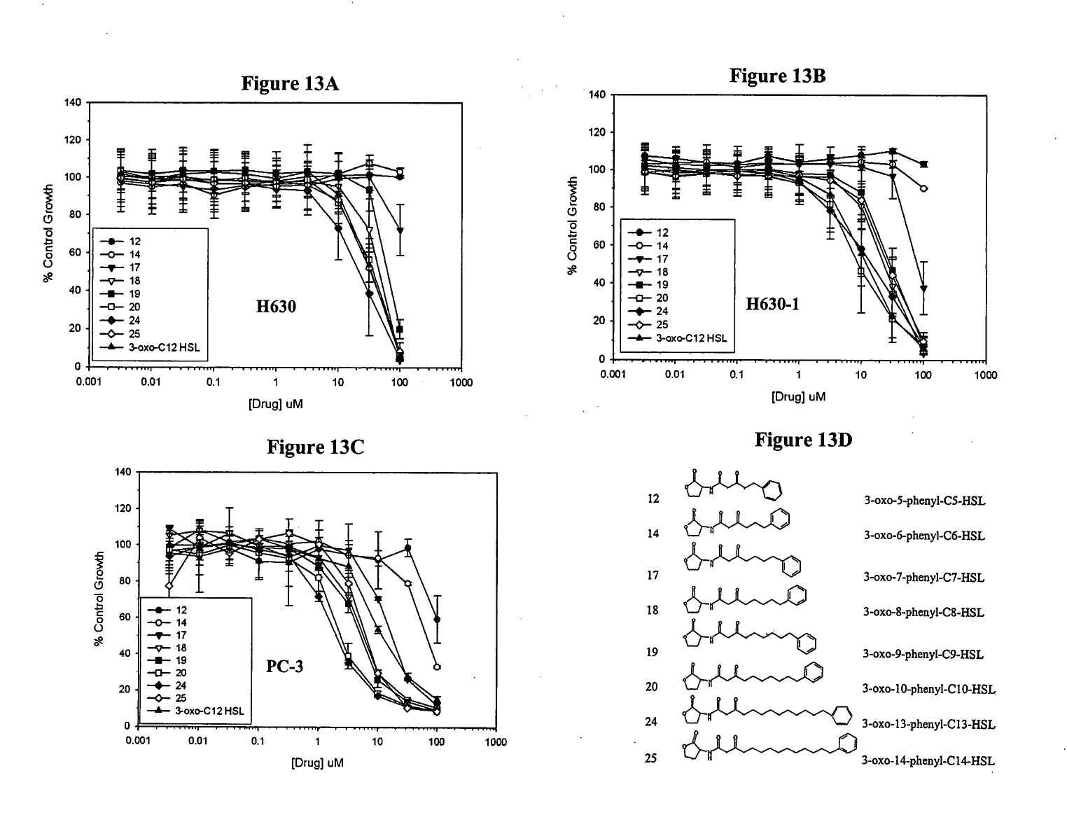

For growth inhibition assays described in this Example, H630, H630-1 and PC-3 (prostate cancer cells) cells were growth in the presence of HSL analog for 5 days. At the end of day 5, cells were fixed and stained with Sulforhodamine-B (SRB), a protein-staining dye. Excess SRB was removed leaving only dye bound to proteins from cells. Protein-bound SRB was then dissolved in base and quantified by spectrophotometric absorbance. In this assay, the amount of SRB bound to protein correlates with the number of cells present, allowing for the analysis of growth of treated versus untreated samples. The HSL analogs are administered at 10 concentrations, spanning a 5 log range. The data is presented in FIGS. 13A-13C and summarized in Table 2.

TABLE 2

| | IC₅₀ (μM) | | |
|---|---|---|---|
| Compound | H630 | H630-1 | PC-3 |
| 12 | >100 | >100 | >100 |
| 14 | >100 | >100 | 80 |
| 17 | >100 | 80 | 20 |
| 18 | 45 | 23 | 6.5 |
| 19 | 60 | 29 | 6 |
| 20 | 35 | 9 | 2.5 |
| 24 | 20 | 15 | 2 |
| 25 | 35 | 26 | 7 |
| 3-oxo-C12 HSL | 35 | 12 | 10 |

The data points in the graphs of FIGS. 13A-13C represent % control growth at a given HSL analog concentration. IC₅₀ values represent the concentration of HSL analog at which growth of the treated cells is inhibited by 50%. The structure of the tested compounds, designated by number, are as set forth in FIG. 13 from which it can be sent that the phenyl-HSL analogs tested range from C5 (compound 12) to C14 (compound 25). The results indicate that generally, in each cancer cell line tested, increasing the alkyl side chain length increases the activity of the compound. This effect decreases as the chain length reaches C14, as indicated by an increase in the IC₅₀ values. These data indicate that acyl chains of up to 14 carbons are effective in the method of the invention, and that the introduction of a phenyl substituent at the terminal position of the alkyl chain may increase the activity of an HSL analog. Specifically, the C10 phenyl analog (compound 20) is equally as active as the 3-oxo-C12 HSL used as a positive control, despite having two fewer carbons in the alkyl chain. The slightly reduced activity of 3-oxo-C12 HSL compared to that shown in the preceding Examples is most likely due to differences in assay conditions.

While this invention has been illustrated by specific embodiments, routine modifications will be apparent to those skilled in the art and such modifications are intended to be within the scope of the invention and the following claims.

1. A method for inhibiting the growth of cancer cells in an individual comprising administering to the individual a composition comprising an L-form acyl homoserine lactone (AHL), wherein the AHL has the formula: 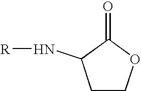
wherein R may be: 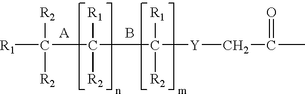
wherein Y may be C═O, C═S, C═NH, CHOH, CHSH, CH₂, or R₁—C—R₂;
R₁ may be independently at each occurrence H, alkyl, Ar or CH₂, wherein Ar is an aromatic substitution;
R₂ may be independently at each occurrence H, F, Cl, Br, or I;
n and m may each independently be between and including 0-10, and the sum of n and m is between and includes 1 and 10; and
A and B designate positions where a group R₃ may be introduced, wherein R₃ is CH═CH;
and wherein administration of the AHL to the individual inhibits the growth of cancer cells.

2. The method of claim 1, wherein the AHL is a 3-oxo-AHL.

3. The method of claim 2, wherein the AHL is a 3-oxo-C12-AHL.

4. The method of claim 1, wherein Ar is a phenol.

5. The method of claim 4, wherein the AHL is selected from the group consisting of 3-oxo-5-phenyl-C5-HSL, 3-oxo-6-phenyl-C6-HSL, 3-oxo-7-phenyl-C7-HSL, 3-oxo-8-phenyl-C8-HSL, 3-oxo-9-phenyl-C9-HSL, 3-oxo-10-phenyl-C10-HSL, 3-oxo-13-phenyl-C13-HSL and 3-oxo-14-phenyl-C14-HSL.

6. The method of claim 1, wherein the composition further comprises a pharmaceutically acceptable carrier.

7. The method of claim 6, wherein the pharmaceutically acceptable carrier is D, L-lactic-co-glycolic acid nanoparticles.

8. The method of claim 1, wherein the cancer cells are colorectal cancer cells, lung cancer cells, breast cancer cells, or prostate cancer cells.

9. The method of claim 6, wherein the composition is administered to the individual by a route selected from the group consisting of intradermal, intramuscular, intraperitoneal, intravenous, subcutaneous, and intranasal

10. The method of claim 6, wherein the composition further comprises a chemotherapeutic agent, wherein the chemotherapeutic agent is a thymidylate synthase (TS) inhibitor or a microtubule inhibitor, and wherein the administration of the AHL in combination with the chemotherapeutic agent enhances the cancer cell growth inhibition activity of the chemotherapeutic agent.

11. The method of claim 10, wherein the chemotherapeutic agent is selected from the group consisting of 5-fluorouracil (FU), fluorodeoxyuridine(FUdR), tomudex, taxol and paclitaxel.

12. The method of claim 11, wherein the chemotherapeutic agent is FU.

13. The method of claim 11, wherein the chemotherapeutic agent is FUDR.

14. The method of claim 11, wherein the chemotherapeutic agent is tomudex.

15. The method of claim 11, wherein the chemotherapeutic agent is taxol.

16. The method of claim 11, wherein the chemotherapeutic agent is paclitaxel.

17. The method of claim 10, wherein the cancer cells are colorectal cancer cells, lung cancer cells, breast cancer cells, or prostate cancer cells.

18. The method of claim 10, wherein the chemotherapeutic agent is a microtubule inhibitor and wherein the cancer cells are resistant to a TS inhibitor.

19. The method of claim 18, wherein the microtubule inhibitor is taxol and the TS inhibitor is FU.

20. The method of claim 10, wherein the composition is administered to the individual by a route selected from the group consisting of intradermal, intramuscular, intraperitoneal, intravenous, subcutaneous, and intranasal.
